(19) United States
(12) Plant Patent Application Publication
(10) Pub. No.: US 2004/0139514 P1
(43) Pub. Date: Jul. 15, 2004

(54) CHRYSANTHEMUM PLANT NAMED 'CHAVINGTROIS'

(76) Inventor: Jean-Pierre Challet, Nuaille (FR)

Correspondence Address: BURNS, DOANE,, SWECKER & MATHIS, L.L.P., P.O. Box 1404, Alexandria, VA, 22313-1404, US

(21) Appl. No.: 10/342,290

(22) Filed: Jan. 15, 2003

(51) Int. Cl. A01H 5/00

(52) U.S. Cl. PLT/263

(57) ABSTRACT

A new and distinct Charm Chrysanthemum cultivar is provided that is the result of a controlled breeding program. Attractive small daisy-like blossoms are formed in profusion in clusters having yellow disc florets and one row of light yellow ray florets. The growth habit is short, bushy, very compact and well-branched with short internodes and yields a generally uniform and spherical overall plant. The foliage has a matte finish, is small and dark green with near white nervures, and contrasts well with the yellow blossom coloration. The response time is approximately eight weeks. The natural flowering time is late-October. When mature the blossoms emit a generous quantity of yellow/orange pollen. The plant grows well in pots.

### BOTANICAL/COMMERCIAL CLASSIFICATION

Ajania pacificum/Decorative Pot Mum

### VARIETAL DENOMINATION

cv. ‘Chavingtrois’

### SUMMARY OF THE INVENTION

The present invention comprises a new and distinct cultivar of Chrysanthemum, botanically known as Ajania pacificum, and hereafter is referred to by the cultivar name ‘Chavingtrois’.

The new cultivar of the present invention was created at Nuaillé, France during the course of a controlled breeding program. The seed parent (i.e., the female parent) was an unnamed plant of the species, and the pollen parent (i.e., the male parent) was designated ‘94/13/9’. Neither parent was patented in the United States. The seeds resulting from the cross were sown and small plants were obtained which were physically and biologically different from each other. Selective study resulted in the identification of a single plant of the new cultivar of the present invention.

It was found that the new Charm Chrysanthemum cultivar of the present invention displays:

(a) a short, bushy, compact, well-branched, and generally spherical growth habit with short internodes,

(b) profusely forms attractive small daisy-like blossoms having yellow disc florets and one row of light yellow ray florets,

(c) forms attractive small dark green foliage having a matte finish with near white nervures,

(d) forms a generous quantity of yellow/orange pollen when the blossoms are mature, and

(e) an ability to grow well in pots.

The new cultivar can be grown singly or in clumps in pots. It also can be grown in the landscape. The yellow blossoms contrast nicely with the dark green foliage. The plant is self-branching and the internodes are short. Pinching is helpful to further enhance branching and produces a large number of shoots; however, such pinching is not necessary since the plant already is inherently well branched. The natural flowering time is late-October.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chavingtrois’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

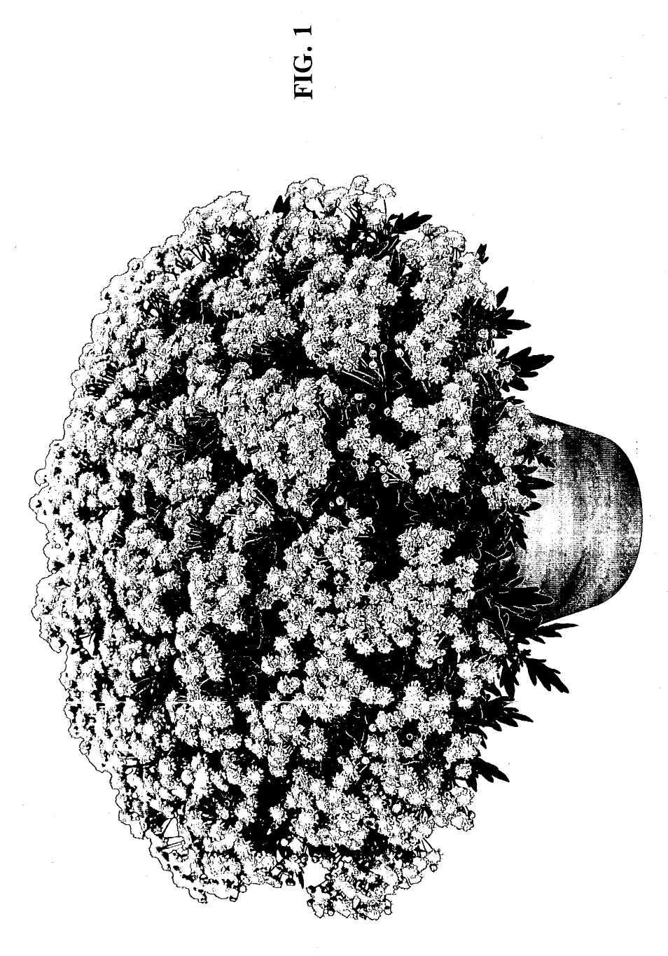

The accompanying FIG. 1 depicts a typical eighteen week-old plant of ‘Chavingtrois’ cultivar while growing in a pot in a greenhouse at Nuaillé, France. The attractive compact spherical growth habit, with a substantially homogenous coverage of yellow blossoms, and dark green foliage with near white nervures are illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General appearance: Short, bushy, and compact with a generally spherical growth habit. The internodes are short and commonly measure approximately 10 to 25 mm on average. When a cutting is placed during week 23 in a four-liter pot, the resulting plant commonly will display a height of approximately 24 to 26 cm on average and a width of approximately 30 to 45 cm on average at the end of flowering during late October.

Foliage: Attractive dark green. The main exposed nervures are near white in coloration.

Flowers: The attractive small daisy-like blossoms are formed in clusters. The flower coverage is substantially homogeneous across the plant. Accordingly, the flowers are so closely grouped as to substantially cover the circumference of the dome-shaped plant. The disc florets are yellow and are surrounded by one row of light yellow ray florets when mature. The flower diameter when mature commonly ranges from approximately 10 to 15 mm. A generous quantity of yellow/orange pollen commonly is displayed in the mature blossoms.

Flower Time: The natural flowering time is late-October.

Response Time: The time between the beginning of the short day period and the flowering date at normal temperature and light intensity is approximately eight weeks.

Usage: Decorative pot mum.

I claim:

1. A new and distinct cultivar of Charm Chrysanthemum plant that displays:
(a) a short, bushy, compact, well-branched, and generally spherical growth habit with short internodes,
(b) profusely forms attractive small daisy-like blossoms having yellow disc florets and one row of light yellow ray florets,
(c) forms attractive small dark green leaves having a matte finish with near white nervures,
(d) forms a generous quantity of yellow/orange pollen when the blossoms are mature, and
(e) an ability to grow well in pots;
substantially as illustrated and described.
